(19) United States
(12) Patent Application Publication
(10) Pub. No.: US 2025/0333807 A1
(43) Pub. Date: Oct. 30, 2025

(54) Recombinant Adeno-Associated Virus (rAAV) Universal Reference Standard for Determining rAAV Genome Copy Titer by Quantitative PCR

(71) Applicant: Agathos Biologics, Fargo, ND (US)

(72) Inventor: Andrzej Noyszewski, Fargo, ND (US)

(21) Appl. No.: 18/649,859

(22) Filed: Apr. 29, 2024

(51) Int. Cl. C12Q 1/70; C12N 15/86

(52) U.S. Cl. CPC C12Q 1/701; C12N 15/86; C12N 2750/14143; C12N 2750/14151; C12N 2830/48; C12N 2830/50; C12Q 2600/166

(57) ABSTRACT

Disclosed is a recombinant adeno-associated virus (rAAV) vector that is a universal reference standard in quantitative PCR assays used to determine the genome copy titer of rAAV-transgene particles. The rAAV-Universal Standard vector includes multiple nucleotide sequences that are targets for PCR assays to support polymerization such as fragments of commonly used promoters, enhancers, polyadenylation sequences, and reporter genes found in rAAV constructs. Those multiple targets ensure that there is a positive reaction and thus provide a standard, positive control and calibrator to assure proper performance of the quantitative PCR assays without the need to use a separate reference standard for each particular rAAV particle assayed. The rAAV-Universal Standard is produced in multiple AAV serotypes and may encode a functional reporter gene to further provide a universal positive control for analytical assays of rAAV-transgene particles.

### STATEMENT REGARDING GOVERNMENT FUNDING

This work was funded in part by Grant Nos. 21-283 and 23-347 from the North Dakota Department of Agriculture's Bioscience Innovation Grant Program.

### SEQUENCE LISTING

An electronic sequence listing (SL 828349-00007.xml; size 12.2 KB; date of creation Apr. 29, 2024) submitted herewith is incorporated by reference in its entirety.

### TECHNICAL FIELD

The invention relates to a new recombinant adeno-associated virus (rAAV) vector that can be used as a universal standard, positive control and calibrator in quantitative PCR assays that determine the genome copy titer of rAAV-transgene particles.

### BACKGROUND OF INVENTION

Genetic medicine holds great potential for correcting disease-causing defects, targeting and destroying cancerous tissues, and providing speed and flexibility for the development of vaccines. Recombinant DNA genetic material to be used as a gene therapy or a vaccine is incorporated into a virus-based vector system, which is produced by expression of the viral vector components in immortalized living cells maintained in tissue culture.

Recombinant adeno-associated virus (rAAV) vectors, which have relatively low immunogenicity, are an important platform for potential gene delivery for the treatment of a variety of human diseases. There is a need to develop clinically-useful rAAV-transgene particles, to optimize genome designs and harness the potential revolutionary biotechnologies that could contribute substantially to the growth of the gene therapy field. Preclinical and clinical successes in AAV-mediated gene replacement and gene editing have helped establish rAAV as a promising therapeutic vector, with four AAV-based therapeutics gaining regulatory approval in Europe or the United States and more in clinical development. Continued study of AAV biology and increased understanding of the associated therapeutic challenges and limitations will build the foundation for future clinical success (see Wang, D., Tai, P. W. L. & Gao, G. Adeno-associated virus vector as a platform for gene therapy delivery. Nat. Rev. Drug. Discov. 18, 358-378 (2019)).

Triple transfection is a method commonly used to produce rAAV particles containing a desired transgene. In that method, an appropriate mammalian cell line that is E1-complementary, i.e., it contains and expresses functional E1 genes of adenovirus, is transfected with a plasmid containing the adenovirus helper genes, a plasmid containing the replication and capsid genes of AAV delivered in trans, and a plasmid with a transgene delivered in cis flanked by the inverted terminal repeats (ITRs) of AAV. Triple transfection results in production of rAAV-transgene particles that, after harvesting and purification, can infect an appropriate host cell and produce the protein encoded by the transgene. Appropriate E1-complementary mammalian cells include HEK293 and AE1-BHK, an E1-complementary baby hamster kidney cell line developed by Agathos Biologics. See agathos.bio/ae1-bhk.

There is an increased demand for production of rAAV-transgene particles at high yield and at large scale for gene delivery and protein expression and production. There is a concomitant need for improved harvesting and purification of rAAV particles, and for improved assays to determine AAV serotype identity and capsid titer, genome copy titer and genome quality (e.g., genomic integrity assays). Quantitative (digital or conventional) polymerase chain reaction (PCR) can be used to determine the genome copy titer of harvested rAAV particles. In digital PCR (such as dPCR), the sample is partitioned into many individual reactions so that either zero, one or more target molecules are present in each reaction, providing improved sensitivity and precision. Sample partitioning allows genomic integrity analysis of a single rAAV genome that is placed in single partition and to evaluate amplification of specified targets. For quantitative dPCR, purified or crude lysate samples of rAAV-transgene particles are prepared using commercially-available protocols, enzymes, e.g., DNase I and Exonuclease I, and buffers as outlined in available kits, e.g., Viral Vector Lysis Kit, (Qiagen, 250272). To prime polymerization during the reaction cycles, practitioners typically use annealing primers that correlate to a portion of the promoter, polyA or transgene sequences of the particular rAAV-transgene particles being assayed. See, e.g., QIAcuity Cell and Gene Therapy (CGT) dPCR Assays (Qiagen, 250236).

Quantitative PCR for determining the genome copy titer of harvested rAAV particles requires use of a positive PCR control to establish the efficiency of the PCR reaction and to assure that the test sample reaction was properly prepared. For determining the rAAV genome copy titer, practitioners use a pre-titered rAAV positive control. The rAAV standard contains a target sequence(s) for PCR, i.e., a sequence that will bind to the annealing primer, which also is being used to amplify the particular rAAV-transgene construct being assayed. Typically, a different rAAV positive control must be used for each separate rAAV-transgene/functional region assayed. There is a need for a rAAV vector that can be used as a universal standard, positive control and calibrator and thereby result in greater efficiency, which in turn will lower cost, and also provide a uniform assay performance for quantitative PCR for determining genome copy titer and assessing genomic integrity of rAAV-transgene particles. The rAAV Universal Reference Standard will further allow standardization (calibration) of quantification methods for multiple targets.

### SUMMARY OF THE INVENTION

One embodiment of the present invention is a recombinant adeno-associated virus (rAAV) universal reference standard for determining rAAV genome copy titer by quantitative PCR. To create the reference standard, an AAV transfer plasmid is synthesized. In one embodiment, the transfer plasmid contains a gene encoding for selection in bacteria, an origin of replication for growth in bacteria, and a rAAV genomic sequence with multiple PCR targets flanked by AAV inverted terminal repeat sequences (ITRs). The gene for selection in bacteria may be any suitable marker for positive or negative selection. In one embodiment, the gene for selection in bacteria encodes expression of a protein that confers resistance to an antibiotic. In a further embodiment, it is a kanamycin-resistance gene. The origin of replication may be any suitable origin of replication for plasmid replication in bacteria. In one embodiment, it is the origin of replication from the ColE1 plasmid.

The multiple PCR targets are nucleic acid fragments found in mammalian or viral promoters, enhancers or polyadenylation sequences associated with gene expression from recombinant vectors. The PCR targets may also be fragments of coding sequences (CDS) of genes commonly used in recombinant vectors. In further embodiments, the multiple PCR targets may be fragments found in a cytomegalovirus (CMV) promoter, a CMV enhancer, a simian virus 40 (SV40) promoter, a SV40 polyadenylation sequence, a bovine growth hormone (bGH) polyadenylation sequence, a human growth hormone (hGH) polyadenylation sequence, a woodchuck hepatitis virus post-transcriptional regulatory element (WPRE), an enhanced green fluorescent protein (eGFP) CDS, a luciferase CDS or a bGH CDS.

The number of PCR targets is limited by the target size necessary to facilitate binding by PCR probes and primers with space between targets. Despite those limitations, the rAAV genome is approximately 4.7 kbp and may accommodate twenty (20) to fifty-seven (57) PCR targets that vary between fragments of approximately 70 bp and approximately 200 bp, which is the size of each PCR target in preferred embodiments. In one embodiment the number of PCR targets is four or more. In a further embodiment, the number of PCR targets is six or more. Each PCR target derives from an independent regulatory element or gene. For example, a fragment from a CMV promoter and a fragment from an eGFP CDS constitute two targets. Two separate fragments from a CMV promoter constitute a single target.

The transfer plasmid is used to produce a rAAV particle that contains a genomic sequence with multiple PCR targets that is used as a universal reference standard for determining genome copy titer by quantitative PCR. The rAAV particle is produced by transfecting a cell line that has a functional E1 gene region of human adenovirus with exogenous nucleic acid including genes for AAV rep/cap proteins, genes for helper proteins and the transfer plasmid described above. Any suitable E1-complementary cell line may be used including HEK293. In a preferred embodiment, the cell line is AE1-BHK. The AAV rep/cap proteins can be AAV serotype 2, AAV serotype 5, AAV serotype 6, AAV serotype 8, a naturally-occurring serotype, an artificial serotype, or a combination of two or more of the foregoing. The helper proteins can be adenovirus helper proteins. In a preferred embodiment, the exogenous nucleic acid consists of three vectors wherein the first vector encodes the gene for AAV rep/cap proteins, a second vector encodes genes for helper proteins and a third vector is the transfer plasmid. The transfected cells are grown under appropriate conditions in appropriate media and the rAAV-Universal Standard particle, of any of the foregoing serotypes and containing the rAAV genomic sequence with multiple PCR targets described above, is harvested.

The rAAV-Universal Standard particle with multiple PCR targets may be used as a universal reference standard, positive control and calibrator for determining genome copy titer of rAAV particles by quantitative PCR. An operator performs a quantitative PCR assay on a first sample containing a rAAV-transgene particle of interest using a PCR primer and probe that will bind to a promoter, enhancer, gene CDS, polyadenylation sequence or other sequence present in the genomic sequence of the rAAV-transgene particle of interest. The rAAV-Universal Standard particle is assayed simultaneously under the same conditions using the same primer and probe used for the particle of interest. At least one of the multiple PCR targets present in the rAAV genomic sequence of the rAAV-Universal Standard particle is bound by the primer and probe. As a result, the rAAV-Universal Standard results a positive PCR assay result. The genome copy titer of the rAAV-Universal Standard is known and, for qPCR, a standard curve can be prepared to for comparison to the test result obtained for the sample of interest. The rAAV-Universal Standard can be used as an instrument and assay calibrator in calibration/verification assay kits. In that manner, the genome copy titer of the sample of interest is determined. The rAAV-Universal Standard particle thus provides a reference standard, positive control and calibrator for determining genome copy titer of rAAV particles by quantitative PCR. In a further embodiment, the rAAV-Universal Standard particle is a reference standard, positive control and calibrator for determining DNA or RNA copy number of nucleic acid-containing vectors by quantitative PCR. The transfer plasmid itself may be used as a universal reference standard for determining genome copy titer of rAAV particles or determining DNA or RNA copy number of nucleic acid-containing vectors by quantitative PCR. Quantitative PCR includes qPCR, dPCR and ddPCR.

In another embodiment of the invention, the rAAV genomic sequence flanked by the ITRs further includes one or more transgenes capable of expressing a functional protein. In a further embodiment, the functional protein is a reporter protein. The reporter protein can be any suitable reporter protein including any suitable fluorescent protein such as eGFP or any suitable luminescent protein such as luciferase.

The inclusion in the rAAV genomic sequence of a gene capable of expressing a functional protein permits the rAAV-Universal Standard particles to be used as a reference standard for an analytical assay demonstrating the expression of the functional protein. In one embodiment, a method of using a rAAV-Universal Standard particle as such a reference consists of infecting host cells with the rAAV-Universal Standard particle, incubating the host cells to allow production of the functional polypeptide encoded by the transgene contained in the rAAV genomic sequence, and perform an analytical assay demonstrating expression of the polypeptide. The host cells can be any appropriate mammalian cell line. In a preferred embodiment, the host cells are HepG2. The functional polypeptide can be any polypeptide whose expression can be measured in an analytical assay. In one embodiment, the functional polypeptide encoded by the transgene is a reporter protein. In a further embodiment, the reporter protein is any suitable fluorescent protein such as eGFP or any suitable luminescent protein such as luciferase.

### DETAILED DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure.

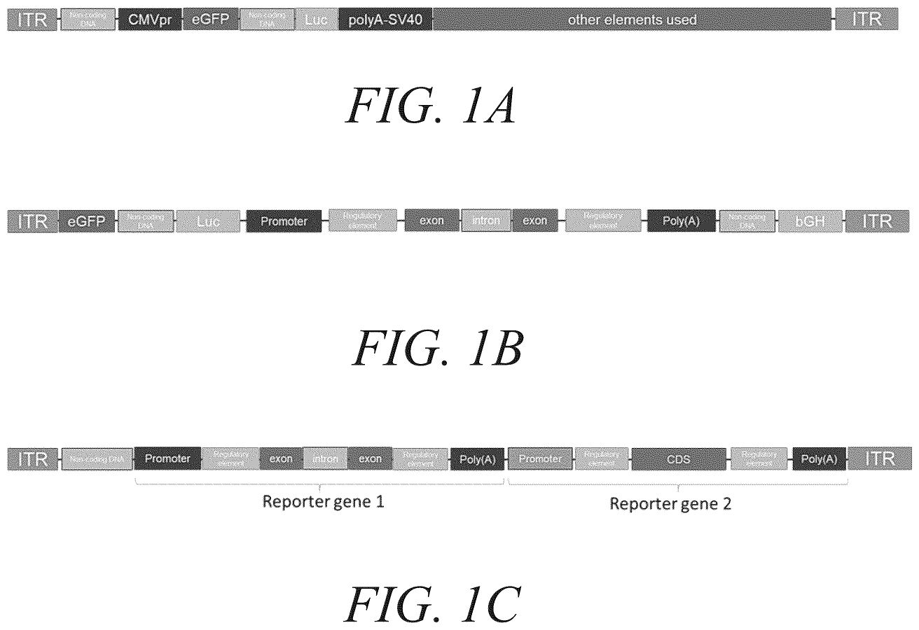

FIG. 1A is a schematic representation of an exemplar of a rAAV-Universal Standard genomic sequence containing multiple target sequences for quantitative PCR placed between ITRs of AAV. The grey regions represent target sequences (e.g., fragments of a CMV promoter, poly (A)-SV40, eGFP CDS and luciferase CDS). Non-coding sequence(s) are placed to extend the length of rAAV genome.

FIG. 1B is a schematic representation of an exemplar of a rAAV-Universal Standard genomic sequence containing multiple target sequences for quantitative PCR and a functional sequence of a reporter gene placed between ITRs of AAV. The grey regions represent target sequences (e.g., fragments of an eGFP CDS, luciferase CDS, promoter etc.). In addition, the “promoter-regulatory sequence-exon-intron-exon-regulatory sequence-poly A” region represents the functional sequences of an unspecified reporter gene. Non-coding DNA sequence(s) are placed to extend the length of rAAV genome.

FIG. 1C is a schematic representation of an exemplar of a rAAV-Universal Standard genomic sequence containing multiple target sequences for quantitative PCR (not shown) and functional sequences of two reporter genes placed between ITRs of AAV. The “promoter-regulatory sequence-exon-intron-exon-regulatory sequence-polyA” regions represent the functional sequences of two unspecified reporter genes. Non-coding DNA sequence(s) are placed to extend the length of rAAV genome.

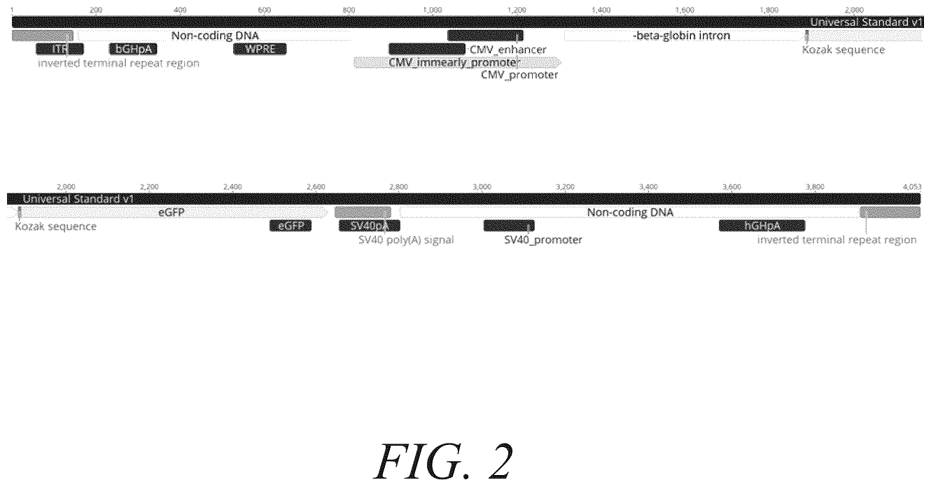

FIG. 2 is a schematic representation of an exemplar of a rAAV-Universal Standard genomic sequence, UStdv1, containing multiple target sequences for quantitative PCR and a functional sequence of a reporter gene placed between ITRs of AAV. The grey regions represent AAV ITRs and fragments of bGHpolyA, WPRE, a CMV promoter, a CMV enhancer, an eGFP CDS, a SV40polyA, a SV40 promoter and a hGHpolyA. It also contains a functional eGFP reporter gene comprising a CMV enhancer, a Kozak sequence, the CDS for eGFP and a SV40polyA sequence. Non-coding DNA sequence(s) are placed to extend the length of rAAV genome.

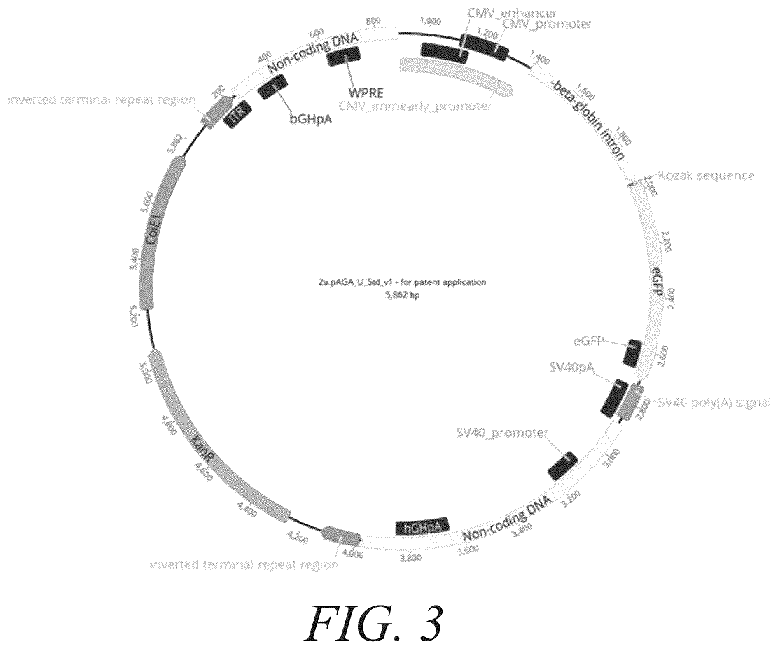

FIG. 3 is a schematic representation of plasmid DNA (pDNA), PAGA-UStdv1, containing the rAAV-Universal Standard genomic sequence, UStdv1, of FIG. 2. The pDNA also contains a kanamycin resistance gene and a ColE1 bacterial origin of replication. Non-coding DNA sequence(s) are placed to extend the length of rAAV genome.

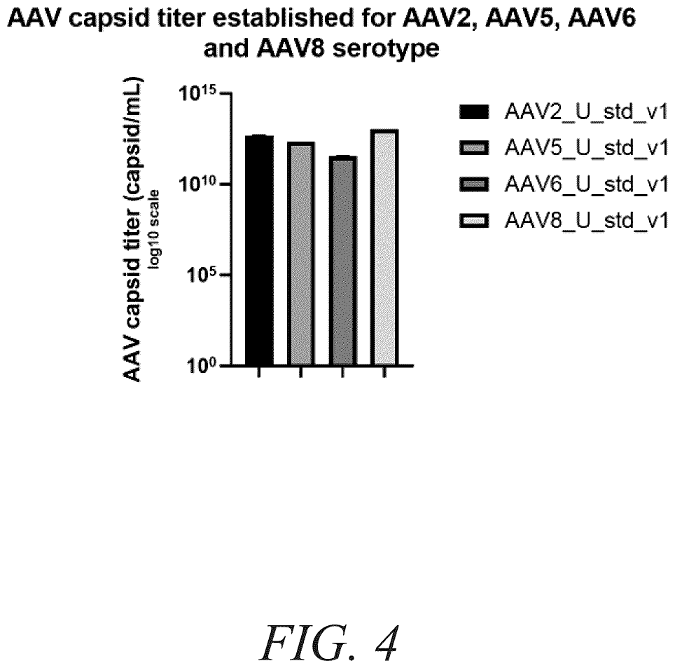

FIG. 4 is a set of bar graphs reporting the production in AE1-BHK cells of rAAV2-UStdv1, rAAV5-UStdv1, rAAV6-UStdv1 and rAAV8-UStdv1 particles as measured in capsids/mL by ELISA. The cells were triple transfected with plasmids containing AAV rep/cap genes for AAV serotype 2, 5, 6 or 8, respectively, adenovirus helper genes and pAGA-UStdv1 of FIG. 3.

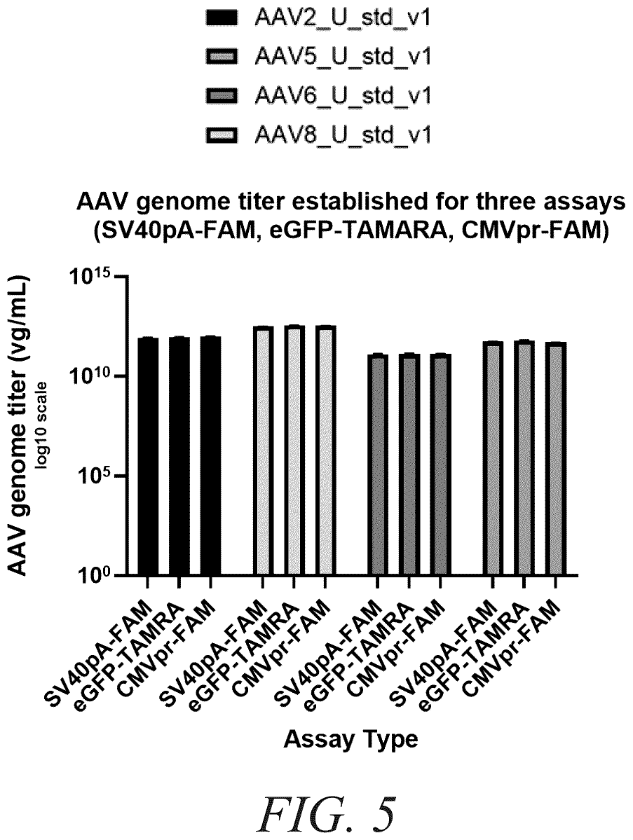

FIG. 5 is a set of bar graphs reporting the titer of rAAV2-UStdv1, rAAV5-UStdv1, rAAV6-UStdv1 and rAAV8-UStdv1 viral genomes/mL produced in AE1-BHK cells as determined by dPCR. The titer of the rAAV-UStdv1 particles was assayed three separate times using PCR primers for the targets SV40polyA region, the coding sequence of eGFP and the CMV promoter. The cells were triple transfected with plasmids containing AAV rep/cap genes for AAV serotype 2, 5, 6 or 8, respectively, adenovirus helper genes and pAGA-UStdv1 of FIG. 3. For each assay, the rAAV sample titer was not normalized prior to testing.

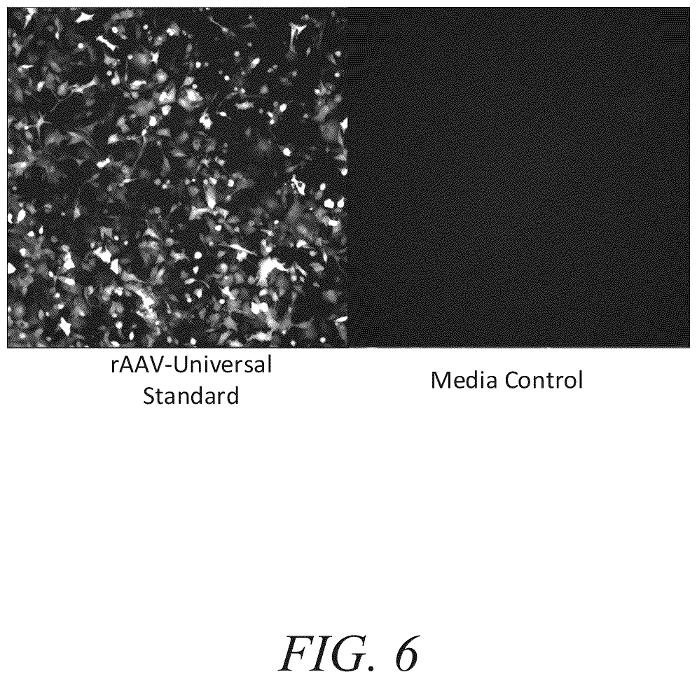

FIG. 6 is a set of images demonstrating transduction by fluorescence in HepG2 cells infected with rAAV8-UStdv1 particles, which contain a functional eGFP transgene (left panel), and a negative control of uninfected HepG2 cells (right panel).

### DETAILED DESCRIPTION OF THE INVENTION

### A. INTRODUCTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In quantitative PCR, which includes qPCR, dPCR and ddPCR, amplification of a target DNA sequence is coupled with quantification of the concentration of that DNA species in the reaction. Quantitative qPCR measures the accumulation of DNA during a PCR reaction. The increase in quantity of DNA at each cycle is measured by the change in intensity of a fluorescent signal. Comparison to a reference sample determines the number of original copies of template DNA in the reaction.

In quantitative digital PCR (dPCR) or quantitative digital droplet (ddPCR), the sample is partitioned into many individual reactions (partitions, droplets) so that either zero, one or more target molecules are present in each reaction. The defining feature of digital PCR is the absolute quantification of nucleic acids. After partitioning, the reactions undergo end-point PCR cycling, and partitions are analyzed for the presence (positive reaction) or absence (negative reaction) of a fluorescent signal. Based on this information, one can calculate the absolute number of molecules present in the sample. Unlike qPCR, dPCR does not rely on standard curves. Consequently, dPCR has a lower detection limit and higher precision than qPCR. See www.qiagen.com/us/applications/digital-pcr?cmpid=CM_PCR_dPCR_Traffic_0123_SEA_GA_NA&gad_source=1&gclid=EAIaIQobChMIw8SoraHvhAMVm0tHAR2W-AdjEAAYAiAAEgJ7ifD_BWE. In dPCR, positive control standards (or calibrators) assure proper design and performance of the assay, assist establishing assays in a new laboratory, and permit comparison of results obtained in different laboratories.

Quantitative PCR is used to determine the genome copy titer of rAAV-transgene particles. The particles may be harvested and purified by known methods. For quantitative PCR of rAAV-transgene particles, the practitioner may use known protocols, enzymes and buffers included in commercially-available kits, e.g., QIAcuity Probe PCR Kit (Qiagen, 250102). Quantitative PCR is used to amplify a target sequence and quantitate the genome copy titer of harvested rAAV-transgene particles. Small amounts of the nucleic acid of the rAAV-transgene particles are recognized and bound during denaturation-renaturation cycles by an annealing primer that is chosen by the practitioner to have a sequence that is anti-sense to that of a promoter, poly (A) or transgene found in the particular rAAV-transgene particle being assayed. The annealing primer is recognized by the polymerase that duplicates the nucleic acid during the polymerization step of the cycle and thereby amplifies the nucleic acid of the rAAV-transgene particle.

The rAAV-Universal Standard vector of the present invention provides a universal standard and positive control for quantitative PCR to determine genome copy titer of rAAV-transgene particles. More broadly, the vector is a universal standard and positive control for quantitative titer determination of any DNA that includes one of the target sequences. Methods for titer determination include qPCR, ddPCR (Biorad, www.bio-rad.com/en-us/life-science/learning-center/introduction-to-digital-pcr/what-is-droplet-digital-pcr#:˜:text=Droplet%20Digital%20PCR%20(ddPCR)%20is,occurs%20in%20each%20individual%20droplet.), dPCR (www.qiagen.com/us/applications/digital-pcr and Thermo www.thermofisher.com/us/en/home/life-science/pcr/digital-pcr.html). The plasmid DNA containing the rAAV-Universal Standard vector is a template for construction of additional standard vectors for assessment and further optimization of quantitative PCR assays and can also serve as a template to assess the performance of quantitative assays not related directly to rAAV viral genome titration. See www.thermofisher.com/us/en/home/life-science/pcr/digital-pcr.html.

The rAAV-Universal Standard vector contains multiple fragments of promoters, enhancers and poly(A) sequences, i.e., PCR targets, used commonly in rAAV-transgene particles. Consequently, regardless of the particular rAAV-transgene construct that is being assayed by PCR, the annealing primer will bind to at least one PCR target and amplify the vector sequence. The rAAV-Universal Standard is used to support quantitative PCR assay design without a need to use multiple separate rAAV standards for each rAAV-transgene particle being titered, such rAAV-transgene constructs typically comprising a specific promoter, a specific polyA sequence and a specific transgene inserted between ITRs. The rAAV-Universal Standard particles are produced in all different AAV serotypes. Inclusion in the Universal Standard of a functional transgene, for example, a functional reporter protein, e.g., eGFP, permits the vector to be used as a positive control for analytical assays related to analytical description of rAAV-transgene particles, e.g., assays to demonstrate and measure production of the protein encoded by the transgene of the rAAV-transgene particles. FIG. 1 demonstrates different possible configurations for the Universal Standard vector. The vector can comprise multiple target sequences (FIG. 1A), multiple target sequences and one coding sequence for a functional gene, for example, a reporter gene (FIG. 1B), or multiple target sequences and multiple coding sequences for multiple different functional genes, including reporter genes (FIG. 1C).

A “vector” is a nucleic acid molecule, a plasmid, virus (e.g., AAV vector), or other vehicle that can be manipulated by insertion or incorporation of a nucleic acid. A viral vector is derived from or based upon one or more nucleic acid elements that comprise a viral genome. The term “recombinant,” as a modifier of vector, such as recombinant AAV vector, as well as a modifier of sequences such as recombinant polynucleotides and polypeptides, means that the compositions have been manipulated (i.e., engineered by recombining genetic sequences) using molecular biology techniques into a form that generally does not occur in nature. Exogenous nucleic acid is nucleic acid originating outside the organism of concern or study.

Adeno-associated virus (AAV) is a small (approximately 25 nm), non-enveloped virus of the Parvoviridae family, including twelve (12) different AAV serotypes, that infects humans and some other primate species. They are replication-deficient and in nature have linear single-stranded DNA (ssDNA) genomes. The wild-type AAV genome is about 4.7 kb and contains rep and cap coding sequences, flanked by ITRs. A “recombinant AAV (rAAV) vector” is derived from the wild type (wt) genome of AAV by using molecular methods to remove all or a portion the wild-type genome from the AAV genome, for example the rep/cap genes, and replacing it with a non-native nucleic acid sequence, referred to as a heterologous nucleic acid or transgene. Typically, one or both inverted terminal repeat (ITR) sequences of the AAV genome are retained and flank the cloned non-native sequence in the AAV vector.

“ITR” means, within the AAV genome, inverted terminal repeat sequences. ITRs serve as the origin of replication and are required for replication, packaging, and vector persistence. They work in conjunction with the viral rep (replication) and cap (capsid) proteins, which bind to the ITRs and initiate replication. ITRs are cis-acting elements, which means that they are in the same DNA molecule as the gene that they package. In a transfer plasmid, the transgene is flanked by two ITRs, and the ITRs are retained with the gene in a rAAV vector. Everything outside the ITRs is left behind.

“bGHpA” means a bovine growth hormone polyadenylation signal. “WPRE” means a woodchuck hepatitis virus posttranscriptional regulatory element. “CMV” means a cytomegalovirus. “eGFP” means an enhanced green fluorescent protein. A “Kozak consensus sequence” is a motif to enhance recognition of a protein translation initiation site. “SV40” means simian virus 40.

The terms “positive control” and “standard” mean, in the context of quantitative PCR, a control template for the polymerization reaction that provides a positive reaction demonstrating that the reaction conditions and assay are correct (e.g., lack of inhibition). The term “calibrator” means, in the context of quantitative PCR, a known concentration that is determined by repetitive testing using a reference or definitive method that spans a reportable range. Calibrators are used to verify performance of the instrument and are used as a part of device qualification.

### B. SEQUENCES

Table 1 provides examples of the nucleotide sequences encoding the genomic sequence of rAAV-UStdv1 and the plasmid pAGA-UStdv1.

TABLE 1
Sequences of the invention.

| | Sequence |
|---|---|
| Sequence Name | Identifier |
| Nucleotide sequence encoding the genomic sequence of | SEQ ID NO: 1 |
| rAAV-UStdv1. | |
| Nucleotide sequence encoding the plasmid, pAGA- | SEQ ID NO: 2 |
| UStdv1. | |

The nucleotide sequence encoding genomic sequence of rAAV-UStdv1 (SEQ ID NO: 1). is displayed in Table 2. below.

TABLE 2
Sequence encoding genomic sequence of the rAAV-UStdv1 (SEQ ID NO: 1).

ttggccactc cctctctgcg cgctcgctcg ctcactgagg ccgggcgacc aaaggtcgcc 60
cgacgcccgg gctttgcccg ggcggcctca gtgagcgagc gagcgcgcag agagggagtg 120
gccaactcca tcactagggg ttcctagatc tgaattcggt accctagtta aaaaaagaga 180
aaaaagtgga aggcacagga ctagagaggc cataacaatg gctgacacct gatatctcga 240
ctgtgccttc tagttgccag ccatctgttg tttgcccctc ccccgtgcct tccttgaccc 300
tggaaggtgc cactcccact gtcctttcct aataaaatga ggagttggaa cactatagca 360
ataataacaa ttagtattat ctctgtaacc ctcacaatca ccctctgaag tatattgtag 420
tgttacctgt ggaaatggag gcaaggaaca gtggactaac tcacccaatg tcacacagcc 480
agtgaggggc acaggtagaa attcaaccca ggcattatgg ctccaactgt ctctttatga 540
ggagttgtgg cccgttgtca ggcaacgtgg cgtggtgtgc actgtgtttg ctgacgcaac 600
ccccactggt tggggcattg ccaccacctg tcagctacta gtgcggatcc aaggagcagc 660
aaaggagacc ttccagggga agctgaattc ttcccttagg tagcaaagag gccctgggac 720
cggggctcgg gggtggtggg taaggaagga ctggcgccca ggtctaagct ccggtatgca 780
gcatcagtgt cctctaagcc atttcccaca taacttacgg taaatggccc gcctggctga 840
ccgcccaacg acccccgccc attgacgtca ataatgacgt atgttcccat agtaacgcca 900
atagggactt tccattgacg tcaatgggtg gagtatttac ggtaaactgc ccacttggca 960
gtacatcaag tgtatcatat gccaagtacg ccccctattg acgtcaatga cggtaaatgg 1020
cccgcctggc attatgccca gtacatgacc ttatgggact ttcctacttg gcagtacatc 1080
tacgtattag tcatcgctat taccatggtg atgcggtttt ggcagtacat caatgggcgt 1140
ggatagcggt ttgactcacg gggatttcca agtctccacc ccattgacgt caatgggagt 1200
ttgttttggc accaaaatca acgggacttt ccaaaatgtc gtaacaactc cgccccattg 1260
acgcaaatgg gcggtaggcg tgtacggtgg gaggtctata taagcagagc tgtgagtttg 1320
gggacccttg attgttcttt ctttttcgct attgtaaaat tcatgttata tggagggggc 1380
aaagttttca gggtgttgtt tagaatggga agatgtccct tgtatcacca tggaccctca 1440
tgataatttt gtttctttca ctttctactc tgttgacaac cattgtctcc tcttattttc 1500
ttttcatttt ctgtaacttt ttcgttaaac tttagcttgc atttgtaacg aatttttaaa 1560
ttcacttttg tttatttgtc agattgtaag tactttctct aatcactttt ttttcaaggc 1620
aatcagggta tattatattg tacttcagca cagttttaga gaacaattgt tataattaaa 1680
tgataaggta gaatatttct gcatataaat tctggctggc gtggaaatat tcttattggt 1740
agaaacaact acaccctggt catcatcctg cctttctctt tatggttaca atgatataca 1800
ctgtttgaga tgaggataaa atactctgag tccaaaccgg gcccctctgc taaccatgtt 1860
catgccttct tctctttcct acaggccacc atggtgagca agggcgagga gctgttcacc 1920
ggggtggtgc ccatcctggt cgagctggac ggcgacgtaa acggccacaa gttcagcgtg 1980
tccggcgagg gcgagggcga tgccacctac ggcaagctga ccctgaagtt catctgcacc 2040
accggcaagc tgcccgtgcc ctggcccacc ctcgtgacca ccctgaccta cggcgtgcag 2100
tgcttcagcc gctaccccga ccacatgaag cagcacgact tcttcaagtc cgccatgccc 2160
gaaggctacg tccaggagcg caccatcttc ttcaaggacg acggcaacta caagacccgc 2220
gccgaggtga agttcgaggg cgacaccctg gtgaaccgca tcgagctgaa gggcatcgac 2280
ttcaaggagg acggcaacat cctggggcac aagctggagt acaactacaa cagccacaac 2340
gtctatatca tggccgacaa gcagaagaac ggcatcaagg tgaacttcaa gatccgccac 2400
aacatcgagg acggcagcgt gcagctcgcc gaccactacc agcagaacac ccccatcggc 2460
gacggccccg tgctgctgcc cgacaaccac tacctgagca cccagtccgc cctgagcaaa 2520
gaccccaacg agaagcgcga tcacatggtc ctgctggagt tcgtgaccgc cgccgggatc 2580
actctcggca tggacgagct gtacaagtac tcagatctcg agctcaagta gccgggtacg 2640
tcgctaactt gtttattgca gcttataatg gttacaaata aagcaatagc atcacaaatt 2700
tcacaaataa agcatttttt tcactgcatt ctagttgtgg tttgtccaaa ctcatcaatg 2760
tatcttatca tgtctggatc cccgcggccg ctcacttgta cagtagtaca gcctttcctt 2820
ctgtggtgct ttctgctgcc tgctgtccca agtgcagcct ccttgtccag gggccctgtt 2880
ctgggggctg gggggtgtga gtaggcggca gggacggagt gggtcagtcg tttcctcccc 2940
tgcctcccag gggccaggat cacagttcca agctgatcat catgcctgaa cttcagggcc 3000
actggcccat cactttggca aagaattggg attcgcgaga attctttggg caacaaacag 3060
tgtagccaag caactccagc catccattct tctatgtcag cagagcctgt agaaccaaac 3120
aatatcagcg ccaccctgcc cccgcgcaca ccccagtgtt ccatgctggg ccctccttag 3180
gagctggaca gcaccaagca gggtggccag gtgttggttg gggggtctgg ggacagagtc 3240
ctctggagag cagccaggga gactggaaat agccagagca ggaaggacat gacgtcagcc 3300
ttcagatgcg ccctgctgat ggggagcaca ggaccaaggc aagggagtga gaccagggct 3360
taattttaga aagtgcgttc tgacagctat atctcaagat gcagtaggtg ggttggggag 3420
gcagtaagct ccccatcctt gggatgttgc atggccactg ggtgggctgg ggtatattat 3480
atagagaaat gacacgtcag atgaggtggc agaggtgtga acaagatggc ctaaaggtag 3540
accccacaat ggccatgtcc ttcctcgctt tgtctgacta ggtgtccttc tataatatta 3600
tggggtggag gggggtggta tggagcaagg ggcaagttgg gaagacaacc tgtagggcct 3660
gcggggtcta ttgggaacca agctggagtg cagtggcaca atcttggctc actgcaatct 3720
ccgcctcctg ggttcaagcg attctcctgc ctcagcctcc cgagttgttg ggattagtgc 3780
atcagtgaac aggcatcaca gaacacttcc tacaggcaca gcccaggcca ggcggcatcc 3840
tgtctccctt atctgtttgt ctgtctgtct tcaggacagg agcttctgga cacctcggct 3900
taagcagaag gaacccctag tgatggagtt ggccactccc tctctgcgcg ctcgctcgct 3960
cactgaggcc gggcgaccaa aggtcgcccg acgcccgggc tttgcccggg cggcctcagt 4020
gagcgagcga gcgcgcagag agggagtggc caa 4053

The nucleotide sequence encoding plasmid DNA. pAGA-UStdv1 (SEQ ID NO: 2). is displayed in Table 3. below.

TABLE 3
Sequence encoding plasmid DNA, PAGA-UStdv1 (SEQ ID NO: 2).

ggatcccggt ccgaagcgcg cggaattcaa aggcctacgt cgacgagctc actagtcgcg 60
gccgctttcg aatctagagt tggccactcc ctctctgcgc gctcgctcgc tcactgaggc 120
cgggcgacca aaggtcgccc gacgcccggg ctttgcccgg gcggcctcag tgagcgagcg 180
agcgcgcaga gagggagtgg ccaactccat cactaggggt tcctagatct gaattcggta 240
ccctagttaa aaaaagagaa aaaagtggaa ggcacaggac tagagaggcc ataacaatgg 300
ctgacacctg atatctcgac tgtgccttct agttgccagc catctgttgt ttgcccctcc 360
cccgtgcctt ccttgaccct ggaaggtgcc actcccactg tcctttccta ataaaatgag 420
gagttggaac actatagcaa taataacaat tagtattatc tctgtaaccc tcacaatcac 480
cctctgaagt atattgtagt gttacctgtg gaaatggagg caaggaacag tggactaact 540
cacccaatgt cacacagcca gtgaggggca caggtagaaa ttcaacccag gcattatggc 600
tccaactgtc tctttatgag gagttgtggc ccgttgtcag gcaacgtggc gtggtgtgca 660
ctgtgtttgc tgacgcaacc cccactggtt ggggcattgc caccacctgt cagctactag 720
tgcggatcca aggagcagca aaggagacct tccaggggaa gctgaattct tcccttaggt 780
agcaaagagg ccctgggacc ggggctcggg ggtggtgggt aaggaaggac tggcgcccag 840
gtctaagctc cggtatgcag catcagtgtc ctctaagcca tttcccacat aacttacggt 900
aaatggcccg cctggctgac cgcccaacga cccccgccca ttgacgtcaa taatgacgta 960
tgttcccata gtaacgccaa tagggacttt ccattgacgt caatgggtgg agtatttacg 1020
gtaaactgcc cacttggcag tacatcaagt gtatcatatg ccaagtacgc cccctattga 1080
cgtcaatgac ggtaaatggc ccgcctggca ttatgcccag tacatgacct tatgggactt 1140
tcctacttgg cagtacatct acgtattagt catcgctatt accatggtga tgcggttttg 1200
gcagtacatc aatgggcgtg gatagcggtt tgactcacgg ggatttccaa gtctccaccc 1260
cattgacgtc aatgggagtt tgttttggca ccaaaatcaa cgggactttc caaaatgtcg 1320
taacaactcc gccccattga cgcaaatggg cggtaggcgt gtacggtggg aggtctatat 1380
aagcagagct gtgagtttgg ggacccttga ttgttctttc tttttcgcta ttgtaaaatt 1440
catgttatat ggagggggca aagttttcag ggtgttgttt agaatgggaa gatgtccctt 1500
gtatcaccat ggaccctcat gataattttg tttctttcac tttctactct gttgacaacc 1560
attgtctcct cttattttct tttcattttc tgtaactttt tcgttaaact ttagcttgca 1620
tttgtaacga atttttaaat tcacttttgt ttatttgtca gattgtaagt actttctcta 1680
atcacttttt tttcaaggca atcagggtat attatattgt acttcagcac agttttagag 1740
aacaattgtt ataattaaat gataaggtag aatatttctg catataaatt ctggctggcg 1800
tggaaatatt cttattggta gaaacaacta caccctggtc atcatcctgc ctttctcttt 1860
atggttacaa tgatatacac tgtttgagat gaggataaaa tactctgagt ccaaaccggg 1920
cccctctgct aaccatgttc atgccttctt ctctttccta caggccacca tggtgagcaa 1980
gggcgaggag ctgttcaccg gggtggtgcc catcctggtc gagctggacg gcgacgtaaa 2040
cggccacaag ttcagcgtgt ccggcgaggg cgagggcgat gccacctacg gcaagctgac 2100
cctgaagttc atctgcacca ccggcaagct gcccgtgccc tggcccaccc tcgtgaccac 2160
cctgacctac ggcgtgcagt gcttcagccg ctaccccgac cacatgaagc agcacgactt 2220
cttcaagtcc gccatgcccg aaggctacgt ccaggagcgc accatcttct tcaaggacga 2280
cggcaactac aagacccgcg ccgaggtgaa gttcgagggc gacaccctgg tgaaccgcat 2340
cgagctgaag ggcatcgact tcaaggagga cggcaacatc ctggggcaca agctggagta 2400
caactacaac agccacaacg tctatatcat ggccgacaag cagaagaacg gcatcaaggt 2460
gaacttcaag atccgccaca acatcgagga cggcagcgtg cagctcgccg accactacca 2520
gcagaacacc cccatcggcg acggccccgt gctgctgccc gacaaccact acctgagcac 2580
ccagtccgcc ctgagcaaag accccaacga gaagcgcgat cacatggtcc tgctggagtt 2640
cgtgaccgcc gccgggatca ctctcggcat ggacgagctg tacaagtact cagatctcga 2700
gctcaagtag ccgggtacgt cgctaacttg tttattgcag cttataatgg ttacaaataa 2760
agcaatagca tcacaaattt cacaaataaa gcattttttt cactgcattc tagttgtggt 2820
ttgtccaaac tcatcaatgt atcttatcat gtctggatcc ccgcggccgc tcacttgtac 2880
agtagtacag cctttccttc tgtggtgctt tctgctgcct gctgtcccaa gtgcagcctc 2940
cttgtccagg ggccctgttc tgggggctgg ggggtgtgag taggcggcag ggacggagtg 3000
ggtcagtcgt ttcctcccct gcctcccagg ggccaggatc acagttccaa gctgatcatc 3060
atgcctgaac ttcagggcca ctggcccatc actttggcaa agaattggga ttcgcgagaa 3120
ttctttgggc aacaaacagt gtagccaagc aactccagcc atccattctt ctatgtcagc 3180
agagcctgta gaaccaaaca atatcagcgc caccctgccc ccgcgcacac cccagtgttc 3240
catgctgggc cctccttagg agctggacag caccaagcag ggtggccagg tgttggttgg 3300
ggggtctggg gacagagtcc tctggagagc agccagggag actggaaata gccagagcag 3360
gaaggacatg acgtcagcct tcagatgcgc cctgctgatg gggagcacag gaccaaggca 3420
agggagtgag accagggctt aattttagaa agtgcgttct gacagctata tctcaagatg 3480
cagtaggtgg gttggggagg cagtaagctc cccatccttg ggatgttgca tggccactgg 3540
gtgggctggg gtatattata tagagaaatg acacgtcaga tgaggtggca gaggtgtgaa 3600
caagatggcc taaaggtaga ccccacaatg gccatgtcct tcctcgcttt gtctgactag 3660
gtgtccttct ataatattat ggggtggagg ggggtggtat ggagcaaggg gcaagttggg 3720
aagacaacct gtagggcctg cggggtctat tgggaaccaa gctggagtgc agtggcacaa 3780
tcttggctca ctgcaatctc cgcctcctgg gttcaagcga ttctcctgcc tcagcctccc 3840
gagttgttgg gattagtgca tcagtgaaca ggcatcacag aacacttcct acaggcacag 3900
cccaggccag gcggcatcct gtctccctta tctgtttgtc tgtctgtctt caggacagga 3960
gcttctggac acctcggctt aagcagaagg aacccctagt gatggagttg gccactccct 4020
ctctgcgcgc tcgctcgctc actgaggccg ggcgaccaaa ggtcgcccga cgcccgggct 4080
ttgcccgggc ggcctcagtg agcgagcgag cgcgcagaga gggagtggcc aacctgcagt 4140
ctcgaggcat gcggtaccaa gcttgtcgag aagtacttgt tacattgcac aagataaaaa 4200
tatatcatca tgaacaataa aactgtctgc ttacataaac agtaatacaa ggggtgttat 4260
gagccatatt caacgggaaa cgtcttgctc aaggccgcga ttaaattcca acatggatgc 4320
tgatttatat gggtataaat gggctcgcga taatgtcggg caatcaggtg cgacaatcta 4380
tcgactttat gggaagcccg atgcgccaga gttgtttctg aaacatggca aaggtagcgt 4440
tgccaatgat gttacagatg agatggtcag actaaactgg ctgacggaat ttatgcctct 4500
tccgaccatc aagcatttta tccgtactcc tgatgatgca tggttactca ccactgcgat 4560
ccccgggaaa acagcattcc aggtattaga agaatatcct gattcaggtg aaaatattgt 4620
tgatgcgctg gcagtgttcc tgcgccggtt gcattcgatt cctgtttgta attgtccttt 4680
taacagcgat cgcgtatttc gtctcgctca ggcgcaatca cgaatgaata acggtttggt 4740
tgatgcgagt gattttgatg acgagcgtaa tggctggcct gttgaacaag tctggaaaga 4800
aatgcataag cttttgccat tctcaccgga ttcagtcgtc actcatggtg atttctcact 4860
tgataacctt atttttgacg aggggaaatt aataggttgt attgatgttg gacgagtcgg 4920
aatcgcagac cgataccagg atcttgccat cctatggaac tgcctcggtg agttttctcc 4980
ttcattacag aaacggcttt ttcaaaaata tggtattgat aatcctgata tgaataaatt 5040
gcagtttcat ttgatgctcg atgagttttt ctaatcagaa ttggttaatt ggttgtaaca 5100
ctggcagagc attacgctga cttgacggga cggcgcctag gctcatgacc aaaatccctt 5160
aacgtgagtt ttcgttccac tgagcgtcag accccgtaga aaagatcaaa ggatcttctt 5220
gagatccttt ttttctgcgc gtaatctgct gcttgcaaac aaaaaaacca ccgctaccag 5280
cggtggtttg tttgccggat caagagctac caactctttt tccgaaggta actggcttca 5340
gcagagcgca gataccaaat actgttcttc tagtgtagcc gtagttaggc caccacttca 5400
agaactctgt agcaccgcct acatacctcg ctctgctaat cctgttacca gtggctgctg 5460
ccagtggcga taagtcgtgt cttaccgggt tggactcaag acgatagtta ccggataagg 5520
cgcagcggtc gggctgaacg gggggttcgt gcacacagcc cagcttggag cgaacgacct 5580
acaccgaact gagataccta cagcgtgagc tatgagaaag cgccacgctt cccgaaggga 5640
gaaaggcgga caggtatccg gtaagcggca gggtcggaac aggagagcgc acgagggagc 5700
ttccaggggg aaacgcctgg tatctttata gtcctgtcgg gtttcgccac ctctgacttg 5760
agcgtcgatt tttgtgatgc tcgtcagggg ggcggagcct atggaaaaac gccagcaacg 5820
cggccttttt acggttcctg gccttttgct ggccttttgc tc 5862

### C. EXAMPLES

The following Examples are exemplary of compositions of matter and methods described herein and should not be considered limiting unless expressly stated.

### Example 1

### Design and Production of rAAV-Universal Standard Vector

Recombinant adeno-associated virus (rAAV)-Universal Standard vectors are designed to include multiple targets for quantitative PCR that correspond to the targets in rAAV-transgene particles of interest. As shown in FIG. 1A-C, exemplary rAAV-Universal Standard genomic sequences include multiple PCR target sequences that include fragments of various promoters, enhancers and polyadenylation sequences including CMV promoter, CMV enhancer, SV40 promoter, SV40pA, bGHpA, hGHpA WPRE regions and other regulatory elements, flanked by ITRs. The length of each PCR target sequence, which typically is a fragment of a promoter or other regulatory sequences or coding sequence of a gene that is about 70 to 200 bp, placed within the rAAV vector is determined by the length necessary to facilitate binding by the primer sequence used in a quantitative PCR assay. The rAAV genome is approximately 4.7 kb, which makes it possible to include a maximum of approximately 20 to 57 unique target sequences in a rAAV-Universal Standard vector depending on the length of each target.

FIG. 1A depicts a rAAV-Universal Standard genomic sequence having, between two AAV ITRs, multiple PCR target sequences including fragments from a CMV promoter, an eGFP CDS, a luciferase CDS and a SV40 polyadenylation region. FIG. 1B depicts a rAAV-Universal Standard genomic sequence having, between two AAV ITRs, multiple PCR target sequences including fragments from an eGFP CDS, a luciferase CDS and a bovine growth hormone CDS. The genomic sequence also includes a functional transgene, depicted as comprising a promoter-regulator element-exon-intron-exon-regulatory element-polyadenylation sequence, which may permit expression of a functional gene including, for example, a reporter gene. FIG. 1C depicts a rAAV-Universal Standard genomic sequence having, between two AAV ITRs, two functional transgenes, each depicted as comprising a promoter-regulator element-exon-intron-exon-regulatory element-polyadenylation sequence, which permits expression of a functional gene including, for example, a reporter gene. Non-coding DNA sequence(s) are placed to extend the length of rAAV genome.

The rAAV-Universal Standard genomic sequences of FIG. 1A-C further include nucleic acid sequences that are not PCR targets, such as random sequences, introns or other non-coding DNA fragments to facilitate optimal viral genome packaging (See Dong et al., Quantitative analysis of the packaging capacity of recombinant adeno-associated virus, Hum. Gen. Ther. 7(17):2101-12 (1996)). The rAAV-Universal Standard sequence is synthesized de novo (GenScript Biotech, USA). Target sequences are identified from publicly-available documentation provided for commercial dPCR assays. See, e.g., Data Sheet dPCR CGT Assay, Qiagen, www.qiagen.com/us/products/discovery-and-translational-research/pcr-qpcr-dpcr/dpcr-assays-kits-and-instruments/dpcr-assays/qiacuity-cell-and-gene-therapy-dpcr-assays. The rAAV-Universal Standard may include a transgene(s) that encodes a functional protein(s) including a functional reporter protein(s) that allows the rAAV-Universal Standard to be a positive control for analytical assays related to analytical description of rAAV-transgene particles. For example, a functional eGFP expressing transgene includes a CMV promoter, a beta-globin intron, a Kozak sequence, an eGFP CDS and SV40 polyA fragments.

FIG. 2 depicts the genomic sequence of rAAV-UStdv1, which contains multiple target sequences for quantitative PCR and one functional reporter gene placed between ITRs of AAV. It contains PCR target sequences including fragments of bGHpolyA, WPRE, a CMV promoter, a CMV enhancer, an eGFP CDS, SV40polyA, SV40 promoter and hGHpolyA. It also contains a functional sequence of the eGFP reporter gene comprising a CMV enhancer, a Kozak sequence, the gene for eGFP and a SV40polyA sequence. The rAAV-UStv1 sequence (SEQ ID NO: 1) was synthesized de novo (GenScript Biotech, USA). FIG. 3 depicts the plasmid DNA (pDNA), PAGA-UStdv1 (SEQ ID NO: 2), containing the rAAV-Universal Standard genomic sequence, UStdv1, of FIG. 2. The pDNA backbone was synthesized de novo (GenScript Biotech, USA) and includes a kanamycin resistance gene and a ColE1 bacterial origin of replication. It includes StuI, SalI, NotI, XbaI, PstI, XhoI, SphI, KpnI, HindIII and other restriction sites positioned to insert the rAAV genomic sequence in the backbone and provide other cloning sites.

### Example 2

### Production of Multiple Serotypes of rAAV-Universal Standard

Eleven serotypes of AAV have been identified, with AAV2 being the best characterized and most commonly used serotype for recombinant technology. There are thermodynamic differences with respect to the capsids of different AAV serotypes. Concern about the possible effect of those differences on results obtained with quantitative PCR means it is desirable to produce the rAAV-Universal Standard in all AAV serotypes. Accordingly, rAAV-UStdv1 particles were produced with the capsid proteins of AAV2, AAV5, AAV6 and AAV8. Quantitative PCR demonstrated that the rAAV-UStdv1 particles can be produced in multiple serotypes and that genome copy titer for rAAV-UStdv1 particles is consistent regardless of the serotype. Four serotypes (AAV2, AAV5, AAV6 and AAV8) of rAAV-UStdv1 particles were produced with the UStdv1 in E1-complementing AE1-BHK cells (Agathos, available at agathos.bio/ae1-bhk/) by the triple transfection method.

### Cell Culture

E1-complementing AE1-BHK cells were cultured in T175 flasks (Thermo Fisher Scientific, Waltham, MA) in DMEM media (ATCC, Manassas, VA) containing 10% FBS (Cytiva, Marlborough, MA) and 1% Pen/Strep (10,000 U/mL Penicillin, 10,000μg/mL Streptomycin) (ATCC, Manassas, VA) and incubated at 37° C. in 5% CO₂ until use.

### Plasmids

Plasmids used for triple transfection include the transfer plasmid prepared in Example 1, pAGA-UStdv1, which contains the rAAV-Universal Standard genomic sequence, Ustdv1. The other plasmids used for triple transfection are commercially available and obtained from Aldevron, Fargo North Dakota (See www.aldevron.com/products/pald-aav) and GeneScript, Piscataway, New Jersey. The rep/cap AAV2 plasmid, pALD-AAV2, is Aldevron catalog number 5057-10, the rep/cap AAV5 plasmid, pALD-AAV5, is Aldevron catalog number 5058-10, the rep/cap AAV6, pALD-AAV6, is Aldevron catalog number 5059-10, and the rep/cap AAV8, PAGA-AAV8, is GeneScript catalog number U38SYNPG0-3. The helper plasmid, pALD-HELP, is Aldevron catalog number 5082-10.

### Triple Transfection of E1-Complementing AE1-BHK Cells

Triple transfection produced rAAV-transgene particles containing the rAAV-UStdv1 as the transgene. The E1-complementary cell line, AE1-BHK, was transfected with three plasmids: 1) a plasmid containing the adenovirus helper genes, 2) a plasmid containing the replication and capsid genes of AAV, and 3) a transfer plasmid pAGA-UStdv1 with the rAAV-UStdv1 flanked by the inverted terminal repeats (ITRs) of AAV. The method is described briefly below.

For each triple transfection, approximately 1×10⁸ AE1-BHK cells were seeded in 5-layer Corning Cell Stacks using 500 mL DMEM supplemented with 10% (v/v) FBS and 1% (v/v) Penicillin/Streptomycin. The flasks were incubated at 37° C. in 5% CO₂ until the cells reached 75-85% confluency. For each flask, two sterile bottles (Corning, NY, various sizes from 125 mL to 500 mL used based on desired volume) were labeled as A and B for preparing the DNA transfection reagent. Bottle A contained three plasmids: 1) the transfer rAAV-UStdv1 plasmid, 2) the helper plasmid, and 3) an AAV rep/cap plasmid of serotype 2, 5, 6, or 8. The amount of each plasmid was calculated as 1 μg of total DNA per one million cells, with a plasmid molar ratio of 1:1:1 diluted in serum free DMEM media. Bottle B contained PEIPro (PEIpro Transfection Reagent REA-245,236 Polyplus, Illkirch-Graffenstaden, France) diluted in serum free DMEM at a concentration three times higher than the plasmid DNA concentration of Bottle A. The contents of tubes A and B were combined and gently mixed by inverting the tube approximately 10 times and vortexing for approximately 10 seconds. The DNA-transfection reagent complex was then incubated at room temperature for at least 10 minutes and no more than 15 minutes.

Before adding the DNA-transfection reagent complex, cells were prepared in DMEM 5% (v/v) FBS media for transfection. Cells were washed with 250 mL of DPBS (Thermo Fisher Scientific, Waltham, MA) and DMEM 5% (v/v) FBS media was added to the cells for a concentration of approximately 1×10⁶ cells/mL. The DNA-transfection reagent complex was added to a 1 L sterile bottle, and media from the cell stack was added into the same 1 L container to mix with the transfection complex. The resulting solution was added back into the cell stack, and the cell stack was equilibrated for equal liquid distribution among the layers. Cells were incubated for 48-72 hours at 37° C. in 5% CO₂.

### Example 3

### Harvesting, Purification and Analysis of rAAV-Universal Standard of Serotypes 2, 5, 6 and 8

Harvesting of rAAV Particles

For harvesting rAAV particles, a freeze-thaw method was employed. Briefly, approximately 48-72 hours after transfection, the transfected cells were detached from flasks by the addition of 0.5 M EDTA for a final EDTA concentration of 50 mM. The cells were incubated for 25-30 minutes at 37° C., with tapping of the flasks to encourage full detachment of the cells. The suspension was collected in 1 L centrifuge bottles and centrifuged at 300×g for 10 minutes at 4° C. Supernatant was collected in a 1 L bottle per centrifuge bottle, leaving the cell pellet. The pellet was resuspended in PBS-MK buffer (1.3 M NaCl, 1 mM MgCl₂, 2.5 mM KCl in PBS, pH 7.4) at a ratio of approximately 10 million cells for every 1 mL of PBS-MK and the sample was vortexed to aid in pellet resuspension. The cells were lysed using a freeze-thaw method: incubation in liquid nitrogen, followed by incubation in a 37° C. water bath, and repetition for a total of three freeze-thaw cycles. The lysed pellet was centrifuged for 3000×g for 20 minutes at 4° C. and filtered through a 0.22 μM Sartorius filter. The rAAV from the supernatant was precipitated by adding 10 g of PEG 8000 (polyethylene glycol) and 5.8 g of NaCl per 100mL of supernatant and stirred at 4° C. until PEG and NaCl were completely dissolved. The solution was stored overnight at 4° C. The solution was centrifuged at 5000×g for 30 mins at 4° C. and the supernatant was discarded. The pellet was resuspended in PBS-MK buffer (500 mL PBS, 101.66 mg MgCl₂ hexahydrate, 93.2 mg KCl) and combined with cell lysate prepared using freeze thaw.

Purification of rAAV Particles

Purification of rAAV particles was performed using AAVX POROS CaptureSelect (Thermo Fisher Scientific) resin, purchased as pre-packed 1 mL columns (Thermo Fisher Scientific, A36652). Columns were used with AKTA Pure 25 M (Cytiva, 29018226) and the purification process was performed at room temperature (approximately 22° C.). The total protein from cell lysate samples was removed as needed by reducing the pH of cell lysate to pH 4 using HCl. After 30 minutes, the pH was adjusted with NaOH to pH 7 and cell lysate was centrifuged at 4000×g for 30 minutes. Cell lysate was filtered using 0.22 μm filters before being loaded on a column. The column was equilibrated with 4 column volumes ([CV]) of 1×PBS (Cytiva, SH30256.02). Cell lysate application was followed by 20 [CV] of 1×PBS (Cytiva, SH30256.02) as the sample application finish step, and additionally with 6 [CV] of 1×PBS (Cytiva, SH30256.02) as a column wash step. The rAAV were eluted with 3 [CV] of low-pH 50 mM Glycine-HCl buffer, pH 2.7 (Polysciences, 24074-1), and collected as three 1 mL fractions. Collection tubes contained 1M Tris-HCl at 1/10 of the fraction volume. Second and third fractions were combined. The collected rAAV samples were buffer exchanged to 1×PBS+0.001% Poloxamer 188 (Gibco, 24040-032) using Amicon Ultracel-2 mL (Merck Millipore, C86533) and filter sterilized using 0.2 μm syringe filters (Thermo Fisher Scientific, 723-2520).

Determination of rAAV Serotype Identity and Capsid Titer

Crude lysate samples of rAAV2-UStdv1 rAAV5-UStdv1, rAAV6-UStdv1 and rAAV8-UStdv1 were tested for the presence of fully assembled viral capsids. Progen AAV2, AAV5, AAV6 and AAV8 Xpress ELISA kits (PRAAV2XP, PRAAV5XP, PRAAV6XP, PRAA8XP) and AAV Titration ELISA kits (PRAAV2R, PRAAV5R, PRAAV6R, PRAAV8R) were used with no deviations to the user manual's protocol (available at us.progen.com/AAV/AAV-ELISA/AII-AAV-ELISA-Products/), and results were read in a Synergy HTX Multi-Mode Reader (BioTek, 1341000). Capsid titer is expressed as capsids/mL (FIG. 4). The results demonstrate successful production of rAAV-UStdv1 particles of multiple AAV serotypes using triple transfection and post-transfection growth in DMEM media containing 5% FBS.

### Example 4

### Genome Copy Titer of rAAV-Universal Standard Determined with Multiple Targets

### Determination of Vector Genome Titer

The production of rAAV2-UStdv1, rAAV5-UStdv1, rAAV6-UStdv1 and rAAV8-UStdv1 was measured by quantitative dPCR. Briefly, the crude lysate samples from triple transfected cells were diluted to 0.1× concentration in 1× phosphate buffered saline (PBS) (VWR, K813-500ML) containing 0.01% Poloxamer 188 (Gibco, 24040-032) and then added to a nucleic acid digestion mixture containing 1× DNase Buffer (New England Biolabs, B0303S), 100U of Deoxyribonuclease I (ThermoFisher, 18047019), 1U of Exonuclease I (ThermoFisher, EN0581), and 0.05% Poloxamer 188. The unencapsidated nucleic acid was digested at 37° C. for 1 hour. DNase-resistant particles were lysed at 95° C. for 15 minutes in a solution containing 10 mM EDTA (ThermoFisher, 15575020), 0.55M NaCl and 0.55% Sarkosyl (Teknova, 2P0355). The treated samples were serially diluted in 1× PCR buffer (ThermoFisher, 4486219) containing 0.05% Poloxamer 188 for AAV genome copy titer analysis by dPCR.

Diluted samples were added to a duplexed dPCR reaction using QIAcuity Probe PCR Kit master mix (Qiagen, 250101). Primers and probes were ordered from IDT and targeted CMV promoter, eGFP and SV40 polyA region with FAM, TAMARA and FAM fluorophores, respectively. Reactions were loaded into a QIAcuity Nanoplate 8.6K plates (Qiagen, 2500011 and/or 250021) and run in a QIAcuity One 5-channel dPCR instrument (Qiagen, 911021). QIAcuity run parameters were set as the default parameters for nanoplate priming and imaging. The onboard thermal cycler profile used an initial denaturation at 95° C. for 15 minutes, followed by 40 cycles of denaturation at 95° C. for 15 seconds and annealing/extension at 60° C. for 30 seconds. AAV titer graphs were prepared in Prism software version 10.2.0 (392) with AAV genome copy titer expressed as log 10 of vg/mL.

An AAV genome copy titer was established in three assays specific to different targets (the SV40 PolyA region, denoted SV40pA-FAM; the coding sequence of eGFP, denoted eGFP-TAMARA; and the CMV promoter, denoted CMVpr-FAM). The titer of each serotype of rAAV-UStdv1 particles was assayed three separate times using PCR primers for the targets SV40polyA region, the coding sequence of eGFP and the CMV promoter. For each assay, the rAAV-UStdv1 was tested from different serotypes: AAV2, AAV5, AAV6, and AAV8. The results shown in FIG. 5 demonstrate that the assay of four different serotype specific rAAV-UStdv1 particles with three separate PCR targets resulted in consistent results for genome copy titer (measured as vg/mL).

### Example 5

### rAAV-Universal Standard is Positive Control for Analytical Studies

The inclusion of a functional reporter gene in the rAAV-Universal Standard permits the vector to serve as a positive control for analytical assays, such as transduction assays. The rAAV8-UStdv1 particles harvested and purified in Example 3, which contain a functional transgene for eGFP, were used to infect HepG2 cells.

For transduction, HepG2 cells were plated in a black, optically clear Phenol view plate (Revvity, 6055302) at approximately 5000 cells/well. Cells were incubated at 37° C. in 5% CO₂ for 72 hrs in 200 μL of DMEM (Corning, 10012CV) in each well. A viral dilution series of rAAV8-usGFP was prepared with concentrations of 2×10⁹, 2×10⁸, and 2×10⁷ vg/mL. The dilutions were made in complete media: DMEM, 10% fetal bovine serum (Cytiva, SH30396.03), 1% Penicillin Streptomycin (ATCC, 302300), with 20 μM etoposide (GoldBio, E9051). Cells were washed with 200 μL of dPBS (Cytiva, SH0028.02) and 200 μL of the appropriate viral dilution was added to the wells in triplicate. A negative control well with only cells and DMEM was prepared. The plate was incubated for 8 days at 37° C. and 5% CO₂. After incubation, the plate was imaged using the Operetta CLS (Revvity). Bright field images were taken along with eGFP filter images (excitation: 460-490 nm, emission: 500-550 nm). The images were taken with the 100× objective. As shown in FIG. 6 (left panel), HepG2 cells infected with the rAAV8-UStdv1 are fluorescent, indicating the presence of a functional eGFP transgene, while the negative control of uninfected HepG2 cells have negligible fluorescence (right panel).

### Example 6

### Use of rAAV-Universal Standard as a Reference Standard and Control

The rAAV Universal Standard may be used as a universal reference standard and positive control for quantitative PCR and thereby verify the proper design and execution of the assay. First, rAAV-Universal Standard particles of a desired serotype are prepared as described herein. Quantitative PCR is performed simultaneously on a sample containing a rAAV-transgene particle of interest and a control containing the rAAV-Universal Standard particle using the same primers, probes, and other experimental conditions. Because the rAAV-Universal Standard contains multiple PCR targets (such as multiple fragments of promoters and polyA sequences), the annealing primer selected to assay the sample rAAV-transgene also binds to at least one target in the control rAAV-Universal Standard particle. A positive PCR result for the control sample containing the rAAV-Universal Standard particle demonstrates that the assay was performed without significant unintentional negative interference, such as contamination or degradation. Further, because the genome copy titer of the control rAAV-Universal Standard particle is known, the result for the rAAV-Universal Standard may be compared to the result for the experimental sample as verification that the assay was properly executed. For qPCR, the results for a series of control rAAV-Universal Standard particles are plotted to create a standard curve used to determine the genome copy titer of the sample rAAV-transgene of interest.

As will be understood by those familiar with the art, the present invention may be embodied in other specific forms without departing from the spirit or other essential characteristics thereof. Accordingly, the disclosures and descriptions herein are intended to be illustrative, but not limiting, of the scope of the invention that is set forth in the following claims.

1. A transfer plasmid for creating a rAAV particle comprising
a gene encoding for selection in bacteria;
an origin of replication for growth in bacteria; and
a rAAV genomic sequence comprising
two AAV inverted terminal repeats (ITRs) that flank
four or more PCR targets.

2. The transfer plasmid of claim 1 in which the ITRs further flank
a transgene encoding a functional protein.

3. The transfer plasmid of claim 2, wherein the functional protein is a reporter protein.

4. The transfer plasmid of claim 3 having at least 90% sequence identity with SEQ ID NO: 2.

5. A rAAV genomic sequence comprising
two AAV ITRs that flank
four or more PCR targets.

6. The rAAV genomic sequence of claim 5 in which the ITRs further flank
a transgene encoding a functional protein.

7. The rAAV genomic sequence of claim 6, wherein the functional protein is a reporter protein.

8. The rAAV genomic sequence of claim 7 having at least 90% sequence identity with SEQ ID NO: 1.

9. A method of producing a rAAV particle that is a universal reference standard for determining genome copy titer comprising:
transfecting a cell line that has a functional E1 gene region of human adenovirus
with exogenous nucleic acid comprising
genes for AAV rep/cap proteins,
genes for helper proteins, and
a transfer plasmid comprising
a gene encoding for selection in bacteria,
an origin of replication for growth in bacteria
a rAAV genomic sequence comprising
two AAV inverted terminal repeats (ITRs) that flank
four or more PCR targets; and
harvesting rAAV particles comprising the rAAV genomic sequence.

10. A rAAV particle that is a universal reference standard for determining genome copy titer comprising:
AAV rep and cap proteins; and
a rAAV genomic sequence comprising
two AAV ITRS that flank
four or more PCR targets.

11. The rAAV particle of claim 10, wherein the four or more PCR targets comprises four or more nucleic acid fragments of
a promoter;
an enhancer;
a polyadenylation sequence; or
a gene sequence.

12. The rAAV particle of claim 11, wherein the four or more PCR targets comprises four or more nucleic acid fragments of
a CMV promoter;
a CMV enhancer;
a SV40 promoter;
a SV40 polyadenylation sequence;
a bGH polyadenylation sequence;
a hGH polyadenylation sequence;
a WPRE;
an eGFP gene;
a luciferase gene; or
a bGH gene.

13. The rAAV particle of claim 12, wherein the AAV rep and cap proteins are
AAV serotype 2.

14. The rAAV particle of claim 13, wherein the two AAV ITRs further flank
a transgene encoding a functional protein.

15. The rAAV particle of claim 14, wherein the functional protein is a reporter protein.

16. The rAAV particle of claim 15, wherein the reporter protein is
eGFP.

17. The rAAV particle of claim 16, wherein the rAAV genomic sequence has at least 90% sequence identity with SEQ ID NO: 1.

18. A method of using a rAAV particle as a universal reference standard for determining genome copy titer of rAAV comprising
performing a quantitative PCR assay on a first sample containing a rAAV particle and a reference sample containing:
a rAAV particle comprising
AAV rep and cap proteins, and
a rAAV genomic sequence comprising
two AAV ITRS that flank
four or more PCR targets; and
determining the genome copy titer of the rAAV particles of the first sample by reference to the genome copy titer of the reference sample.

19. The method of claim 18, wherein four or more PCR targets comprise four or more nucleic acid fragments of
a promoter;
an enhancer;
a polyadenylation sequence; or
a gene sequence.

20. The method of claim 19, wherein the four or more PCR targets comprises four or more nucleic acid fragments of
a CMV promoter;
a CMV enhancer;
a SV40 promoter;
a SV40 polyadenylation sequence;
a bGH polyadenylation sequence;
a hGH polyadenylation sequence;
a WPRE;
an eGFP gene;
a luciferase gene; or
a bGH gene.

21. The method of claim 20, wherein the AAV rep and cap proteins are
AAV serotype 2.

22. The method of claim 21, wherein the two AAV ITRs further flank
a transgene encoding a functional protein.

23. The method of claim 22, wherein the functional protein is a reporter protein.

24. The method of claim 23, wherein the reporter protein is
eGFP.

25. The method of claim 24, wherein the rAAV genomic sequence has at least 90% sequence identity with SEQ ID NO: 1.

26. A method of using a rAAV particle as a reference standard for an analytical assay demonstrating the expression of a polypeptide encoded by a reporter gene comprising
infecting host cells with the rAAV particle of claim 14;
incubating the host cells to allow production of the polypeptide encoded by the reporter gene; and
performing an analytical assay demonstrating expression of the polypeptide encoded by the reporter gene.

27. The method of claim 26, wherein the host cell is HepG2.

28. The method of claim 27, wherein the polypeptide encoded by the reporter gene is eGFP.


SEQUENCE LISTING (2 sequences)

SEQ ID NO: 1 - DNA, 4053 bp, synthetic construct
  source 1..4053: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttaaaaaaagagaaaaaagtggaaggcacaggactagagaggccataacaatggctgacacctgatatctcgactgtgccttctagttgccagccatctgttgtttgcccctcccccgtgccttccttgaccctggaaggtgccactcccactgtcctttcctaataaaatgaggagttggaacactatagcaataataacaattagtattatctctgtaaccctcacaatcaccctctgaagtatattgtagtgttacctgtggaaatggaggcaaggaacagtggactaactcacccaatgtcacacagccagtgaggggcacaggtagaaattcaacccaggcattatggctccaactgtctctttatgaggagttgtggcccgttgtcaggcaacgtggcgtggtgtgcactgtgtttgctgacgcaacccccactggttggggcattgccaccacctgtcagctactagtgcggatccaaggagcagcaaaggagaccttccaggggaagctgaattcttcccttaggtagcaaagaggccctgggaccggggctcgggggtggtgggtaaggaaggactggcgcccaggtctaagctccggtatgcagcatcagtgtcctctaagccatttcccacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagccgggtacgtcgctaacttgtttattgcagcttataatggttacaaataaagcaatagcatcacaaatttcacaaataaagcatttttttcactgcattctagttgtggtttgtccaaactcatcaatgtatcttatcatgtctggatccccgcggccgctcacttgtacagtagtacagcctttccttctgtggtgctttctgctgcctgctgtcccaagtgcagcctccttgtccaggggccctgttctgggggctggggggtgtgagtaggcggcagggacggagtgggtcagtcgtttcctcccctgcctcccaggggccaggatcacagttccaagctgatcatcatgcctgaacttcagggccactggcccatcactttggcaaagaattgggattcgcgagaattctttgggcaacaaacagtgtagccaagcaactccagccatccattcttctatgtcagcagagcctgtagaaccaaacaatatcagcgccaccctgcccccgcgcacaccccagtgttccatgctgggccctccttaggagctggacagcaccaagcagggtggccaggtgttggttggggggtctggggacagagtcctctggagagcagccagggagactggaaatagccagagcaggaaggacatgacgtcagccttcagatgcgccctgctgatggggagcacaggaccaaggcaagggagtgagaccagggcttaattttagaaagtgcgttctgacagctatatctcaagatgcagtaggtgggttggggaggcagtaagctccccatccttgggatgttgcatggccactgggtgggctggggtatattatatagagaaatgacacgtcagatgaggtggcagaggtgtgaacaagatggcctaaaggtagaccccacaatggccatgtccttcctcgctttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattagtgcatcagtgaacaggcatcacagaacacttcctacaggcacagcccaggccaggcggcatcctgtctcccttatctgtttgtctgtctgtcttcaggacaggagcttctggacacctcggcttaagcagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 5862 bp, synthetic construct
  source 1..5862: mol_type = other DNA
ggatcccggtccgaagcgcgcggaattcaaaggcctacgtcgacgagctcactagtcgcggccgctttcgaatctagagttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttaaaaaaagagaaaaaagtggaaggcacaggactagagaggccataacaatggctgacacctgatatctcgactgtgccttctagttgccagccatctgttgtttgcccctcccccgtgccttccttgaccctggaaggtgccactcccactgtcctttcctaataaaatgaggagttggaacactatagcaataataacaattagtattatctctgtaaccctcacaatcaccctctgaagtatattgtagtgttacctgtggaaatggaggcaaggaacagtggactaactcacccaatgtcacacagccagtgaggggcacaggtagaaattcaacccaggcattatggctccaactgtctctttatgaggagttgtggcccgttgtcaggcaacgtggcgtggtgtgcactgtgtttgctgacgcaacccccactggttggggcattgccaccacctgtcagctactagtgcggatccaaggagcagcaaaggagaccttccaggggaagctgaattcttcccttaggtagcaaagaggccctgggaccggggctcgggggtggtgggtaaggaaggactggcgcccaggtctaagctccggtatgcagcatcagtgtcctctaagccatttcccacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctgtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacaggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagccgggtacgtcgctaacttgtttattgcagcttataatggttacaaataaagcaatagcatcacaaatttcacaaataaagcatttttttcactgcattctagttgtggtttgtccaaactcatcaatgtatcttatcatgtctggatccccgcggccgctcacttgtacagtagtacagcctttccttctgtggtgctttctgctgcctgctgtcccaagtgcagcctccttgtccaggggccctgttctgggggctggggggtgtgagtaggcggcagggacggagtgggtcagtcgtttcctcccctgcctcccaggggccaggatcacagttccaagctgatcatcatgcctgaacttcagggccactggcccatcactttggcaaagaattgggattcgcgagaattctttgggcaacaaacagtgtagccaagcaactccagccatccattcttctatgtcagcagagcctgtagaaccaaacaatatcagcgccaccctgcccccgcgcacaccccagtgttccatgctgggccctccttaggagctggacagcaccaagcagggtggccaggtgttggttggggggtctggggacagagtcctctggagagcagccagggagactggaaatagccagagcaggaaggacatgacgtcagccttcagatgcgccctgctgatggggagcacaggaccaaggcaagggagtgagaccagggcttaattttagaaagtgcgttctgacagctatatctcaagatgcagtaggtgggttggggaggcagtaagctccccatccttgggatgttgcatggccactgggtgggctggggtatattatatagagaaatgacacgtcagatgaggtggcagaggtgtgaacaagatggcctaaaggtagaccccacaatggccatgtccttcctcgctttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattagtgcatcagtgaacaggcatcacagaacacttcctacaggcacagcccaggccaggcggcatcctgtctcccttatctgtttgtctgtctgtcttcaggacaggagcttctggacacctcggcttaagcagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacctgcagtctcgaggcatgcggtaccaagcttgtcgagaagtacttgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgactttatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcctaggctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc